(19) United States
(12) Patent Application Publication
(10) Pub. No.: US 2014/0174906 A1
(43) Pub. Date: Jun. 26, 2014

(54) PHOTOCATALYTIC SYSTEM FOR THE REDUCTION OF CARBON DIOXIDE

(71) Applicant: SUNPOWER TECHNOLOGIES LLC, San Marcos, CA (US)

(72) Inventor: DANIEL LANDRY, REDONDO BEACH, CA (US)

(73) Assignee: SUNPOWER TECHNOLOGIES LLC, SAN MARCOS, CA (US)

(21) Appl. No.: 13/722,476

(22) Filed: Dec. 20, 2012

(51) Int. Cl. B01J 35/00; B01J 23/30; B01J 23/36; C07C 1/22; B01J 27/04

(52) U.S. Cl. CPC B01J 35/004; C07C 1/22; B01J 27/04; B01J 23/36; B01J 23/30; B82Y 30/00
USPC 204/157.15; 502/100; 502/216; 502/300; 502/305; 977/902; 977/774; 977/810

(57) ABSTRACT

A system and method employing sunlight energy for the reduction of carbon dioxide into methane and water are disclosed. Methane gas may then be stored for later use as fuel. The system and method may use inorganic capping agents that cap the surface of semiconductor nanocrystals to form photocatalytic capped colloidal nanocrystals, which may be deposited on a substrate and treated to form a photoactive material. The photoactive material may be employed in the system to harvest sunlight and produce energy necessary for carbon dioxide reduction. The system may also include elements necessary to collect and transfer methane, for subsequent transformation into electrical energy.

### BACKGROUND

1. Technical Field

The present disclosure relates in general to carbon dioxide (CO₂) reduction systems, and more specifically, to compositions and methods for forming photocatalytic capped colloidal nanocrystals.

2. Background

The extensive use of fossil fuels over the past century has led to a significant increase in the amount of carbon dioxide in the atmosphere. Carbon dioxide is the most abundant greenhouse gas and one of the main contributors to global climate change. Its increase has mandated intensive efforts to develop alternative energy sources, including considerable effort directed towards the utilization of solar energy.

According to the American Energy Information Administration (EIA) and to the International Energy Agency (IEA), the world-wide energy consumption will on average continue to increase by 2% per year. That increase doubles energy consumption every 35 years. Renewable energy resources such as wind, tidal, geothermal, nuclear, biomass, and hydroelectric are unlikely to provide sufficient amount of energy. By contrast, the sun produces 10×10¹⁵ TW of clean energy that reaches the surface of the earth, of which around 600 TW can be utilized.

Enormous efforts have been recently attracted to seek new materials and/or novel structures for efficient solar energy conversions. To be economically competitive, solar energy needs to be converted into other forms that can be directly utilized with high efficiency and low cost.

One enticing topic in this broad endeavor is the solar photocatalytic reduction of carbon dioxide to various higher energy products so as to store solar energy as chemical energy and create renewable fuels. Similarly, transforming carbon dioxide into a source of fuel could offer an attractive way to decrease atmospheric concentrations. One way to accomplish this conversion is through the light-driven reduction of carbon dioxide to methane (CH₄) or methanol (CH₃OH). One advantage is the existing infrastructure which already supports the delivery of natural gas and liquid fuels, which makes these possible CO₂ reduction products particularly appealing.

Methane is the main component of natural gas. The combustion of methane is highly exothermic. The energy released by the combustion of methane, in the form of natural gas, is used directly to heat homes and commercial buildings. It is also used in the generation of electric power, and when compressed, employed as a vehicle fuel. During the past decade natural gas accounted for about ⅕ of the total energy consumption worldwide, and about ⅓ in the United States.

As a residential fuel, methane is burned in furnaces, water heaters, cooking stoves, and clothes dryers. As an industrial fuel, it is burned in kilns (special furnaces) used to bake bricks and ceramic tiles and to produce cement. Natural gas is also used for generating steam in water boilers and as a source of heat in glass making and food processing.

Likewise, methane serves as a raw material for creating petrochemicals, which are chemicals that are specifically derived from natural gas or petroleum. In turn, petrochemicals are used as a base product for making fertilizers, detergents, pharmaceuticals, plastics, and numerous other goods.

### Carbon Dioxide Reduction Employing Nanosized Photocatalysts

The principles of photocatalytic carbon dioxide reduction require high surface areas for electron excitation and collection, and the use of nanocatalysts with high surface to volume ratio is a favorable match. Semiconductor nanocrystals can improve photocatalysis through the combined effects of quantum confinement and unique surface morphologies. Surface modification of nanosized catalysts may affect redox potentials, and may be used to enhance the efficiency of charge transfer and charge separation.

Nanometer-scaled composites provide the opportunity to combine useful attributes of two or more materials within a single composite. Alternately, one may generate entirely new properties as a result of the intermixing of two or more materials. Semiconductor nanocrystals also provide an improved degree of electronic and structural flexibility, primarily exemplified by the ability to continuously tailor the size of the particles and therefore, via quantum confinement effects, the electronic properties of the particles. An appropriately-tailored inorganic nanocomposite may provide outstanding thermoelectric characteristics. Inorganic nanocomposites may also exhibit high tunability.

Useful properties can be expected as a result of the nanometer scale integration of inorganic components. Several useful examples of inorganic nanocomposites include an intimate network of n- and p-type semiconductors. The n- and p-type semiconductors should have appropriate choice of band gaps and offsets. The resulting array of distributed p-n junctions would be useful for solar cell technology.

There still exists a need for improvement in this field, including the need for development of improved materials and devices that may operate with higher energy conversion efficiency for alternative fuel generation. A solar energy based technology to recycle carbon dioxide into readily transportable hydrocarbon fuel may reduce atmospheric carbon dioxide levels and may partly fulfill energy demands within the existing hydrocarbon based fuel economy.

### SUMMARY

An aspect of the current disclosure is a method for reducing carbon dioxide employing photocatalytic capped colloidal nanocrystals including first and second semiconductor nanocrystals. First semiconductor nanocrystal may be capped with first inorganic capping agent and may be employed as a reduction photocatalyst, while second semiconductor nanocrystal may be capped with second capping agent and may be employed as an oxidation photocatalyst. Semiconductor nanocrystals may be configured in different shapes such as tetrapod, spherical, core/shell, carbon nanotubes and nanorods. Examples of photocatalytic capped colloidal nanocrystals may include noble metals, nickel, copper, titanium dioxide, zinc sulfide and mixtures thereof.

In order to form a photoactive material, photocatalytic capped colloidal nanocrystals may be applied onto a suitable porous substrate having a pore size sufficient to admit CO₂ and H₂ gas passage. Photoactive material may be placed inside a reaction vessel where carbon dioxide and hydrogen gas are introduced. Light from a light source, such as sunlight, enters reaction vessel so that a redox reaction may take place between photoactive material, carbon dioxide and hydrogen. Suitable light source may have a wavelength between 300 nm to about 1500 nm.

The methane gas produced with the present method, may be easily delivered as fuel for homes, businesses, and factories. Methane is also a basic raw material for many compounds which may be employed to produce thousands of products of everyday use, such as plastics. Therefore, the use of methane may help to decrease fossil fuel dependency.

### BRIEF DESCRIPTION OF THE DRAWINGS

Embodiments of the present invention are described by way of example with reference to the accompanying figures which are schematic and not intended to be drawn to scale.

FIG. 1 shows a flow diagram of a method of forming a composition of photocatalytic capped colloidal nanocrystals.

FIG. 2 depicts an illustrative embodiment of a spherical configuration of a photocatalytic capped colloidal nanocrystal.

FIG. 3 shows an illustrative embodiment of a tetrapod configuration of a photocatalytic capped colloidal nanocrystal.

FIG. 4 depicts an illustrative embodiment of a core/shell configuration of a photocatalytic capped colloidal nanocrystal.

FIG. 5 shows an illustrative embodiment of a carbon nanotube configuration of a photocatalytic capped colloidal nanocrystal.

FIG. 6 depicts an illustrative embodiment of a photocatalytic capped colloidal nanocrystal in nanorod configuration.

FIG. 7 depicts an embodiment of spraying deposition method and annealing method used to apply and treat photocatalytic capped colloidal nanocrystals on a substrate.

FIG. 8 shows a photoactive material employed in the present disclosure.

FIG. 9 represents the carbon dioxide reduction process taking place in a reaction vessel.

FIG. 10 illustrates the charge separation process that may occur during carbon dioxide reduction process.

FIG. 11 shows the chemical reactions that describe carbon dioxide reduction process.

FIG. 12 represents a carbon dioxide reduction system employing carbon dioxide reduction process.

### DETAILED DESCRIPTION

### Definitions

As used here, the following terms have the following definitions:

“Carbon dioxide reduction” refers to the conversion of carbon dioxide to useful chemicals such as hydrocarbons or synthesis gas by reacting carbon dioxide with hydrogen/hydrogen containing compounds.

“Semiconductor nanocrystals” refers to particles sized between about 1 and about 100 nanometers made of semiconducting materials.

“Electron-hole pairs” refers to charge carriers that are created when an electron acquires energy sufficient to move from a valence band to a conduction band and creates a free hole in the valence band, thus starting a process of charge separation.

“Inorganic capping agent” refers to semiconductor particles that cap semiconductor nanocrystals.

“Photoactive material” refers to a substance capable of a chemical or physical change in response to light.

“Nanocrystal growth” refers to a synthetic process including the reacting of component precursors of a semiconductor crystal in the presence of a stabilizing organic ligand, taking into account process parameters in order to control the growth and physical or chemical properties of the nanocrystal.

### Method for Forming Composition of Photocatalytic Capped Colloidal Nanocrystals

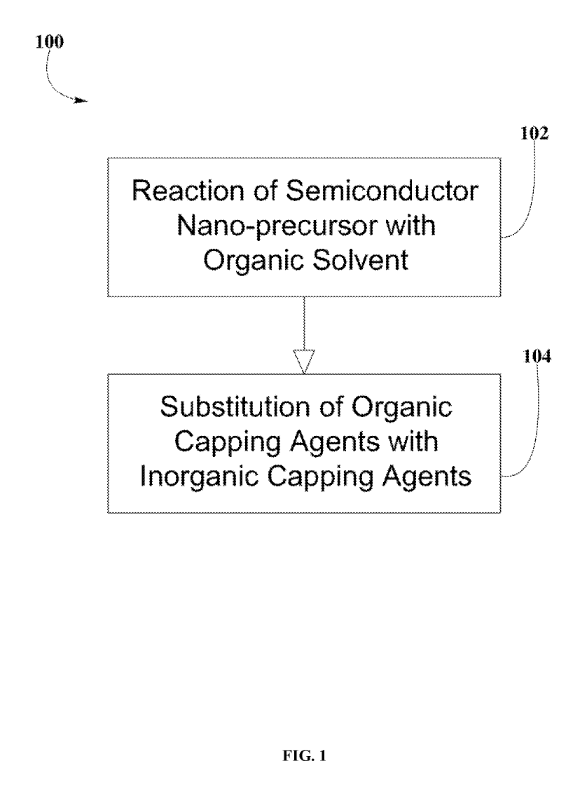

FIG. 1 shows a flow diagram of a method 100 for forming a composition of photocatalytic capped colloidal nanocrystals. Photocatalytic capped colloidal nanocrystals may be synthesized following accepted protocols, and may include one or more semiconductor nanocrystals and one or more inorganic capping agents.

To synthesize photocatalytic capped colloidal nanocrystals, semiconductor nanocrystals are first grown by reacting semiconductor nanocrystal precursors in the presence of an organic solvent 102. Here, the organic solvent may be a stabilizing organic ligand, referred to as organic capping agent. One example of an organic capping agent may be trioctylphosphine oxide (TOPO). TOPO 99% may be obtained from Sigma-Aldrich Co. LLC (St. Louis, Mo.). TOPO capping agent prevents the agglomeration of semiconductor nanocrystals during and after their synthesis. Additionally, the long organic chains radiating from organic capping agents on the surface of a semiconductor nanocrystal may assist in the suspension and/or solubility of a semiconductor in a solvent. Other suitable organic capping agents may include long-chain aliphatic amines, long-chain aliphatic phosphines, long-chain aliphatic carboxylic acids, long-chain aliphatic phosphonic acids and mixtures thereof.

Examples of semiconductor nanocrystals may include the following: Ag, Au, Ru, Rh, Pt, Pd, Os, Ir, Ni, Cu, CdS, Pt-tipped, TiO₂, Mn/ZnO, ZnO, CdSe, SiO₂, ZrO₂, SnO₂, WO₃, MoO₃, CeO₂, ZnS, WS₂, MoS₂, SiC, GaP, Cu—Au, Ag, and mixtures thereof; Cu/TiO2, Ag/TiO2, Cu—Fe/TiO2-SiO2 and dye-sensitized Cu—Fe/P25 coated optical fibers.

The chemistry of capping agents may control several system parameters. For example, varying the size of semiconductor nanocrystals may often be achieved by changing the reaction time, reaction temperature profile, or structure of the organic capping agent used to passivate the surface of a semiconductor nanocrystal during growth. Other factors may include growth rate or shape, the dispersability in various solvents and solids, and even the excited state lifetimes of charge carriers in semiconductor nanocrystals. The flexibility of synthesis is demonstrated by the fact that often one capping agent may be chosen for its growth control properties, and then later a different capping agent may be substituted to provide a more suitable interface or to modify optical properties or charge carrier mobility. As known in the art, a number of synthetic routes for growing semiconductor nanocrystals may be employed, such as a colloidal route, as well as high-temperature and high-pressure autoclave-based methods. In addition, traditional routes using high temperature solid state reactions and template-assisted synthetic methods may be employed.

The morphologies of semiconductor nanocrystals may include nanocrystals, nanorods, nanoplates, nanowires, dumbbell-like nanoparticles, and dendritic nanomaterials. Each morphology may include an additional variety of shapes such as spheres, cubes, tetrahedra (tetrapods), among others. Neither the morphology nor the size of a semiconductor nanocrystal inhibits method 100; rather, the selection of morphology and size of semiconductor nanocrystals may permit the tuning and control of the properties of photocatalytic capped colloidal nanocrystals.

In alternative embodiments seeking to modify optical properties as well as to enhance charge carriers mobility, semiconductor nanocrystals may be capped by inorganic capping agents in polar solvents instead of organic capping agents. In those embodiments, inorganic capping agents may act as photocatalysts to facilitate a photocatalytic reaction on the surface of semiconductor nanocrystals. Optionally, semiconductor nanocrystals may be modified by the addition of not one but two different inorganic capping agents. In that instance, a reduction inorganic capping agent is first employed to facilitate the reduction half-cell reaction; then, an oxidation inorganic capping agent facilitates the oxidation half-cell reaction.

Inorganic capping agents may take many forms. In some embodiments these agents may be neutral or ionic, or they may be discrete species, either linear or branched chains, or two-dimensional sheets. Ionic inorganic capping agents are commonly referred to as salts, pairing a cation and an anion. The portion of the salt specifically referred to as an inorganic capping agent is the ion that displaces the organic capping agent.

A further embodiment involves substitution of organic capping agents with inorganic capping agents 104. There, organic capped semiconductor nanocrystals in the form of a powder, suspension, or a colloidal solution, may be mixed with inorganic capping agents, causing a reaction of organic capped semiconductor nanocrystals with inorganic capping agents. This reaction rapidly produces insoluble and intractable materials. Then, a mixture of immiscible solvents may be used to control the reaction, facilitating a rapid and complete exchange of organic capping agents with inorganic capping agents. During this exchange, organic capping agents are released.

Generally, inorganic capping agents may be dissolved in a polar solvent, while organic capped semiconductor nanocrystals may be dissolved in an immiscible, generally non-polar, solvent. These two solutions may then be combined and stirred for about 10 minutes, after which a complete transfer of semiconductor nanocrystals from the non-polar solvent to the polar solvent may be observed. Immiscible solvents may facilitate a rapid and complete exchange of organic capping agents with inorganic capping agents.

Organic capped semiconductor nanocrystals may react with inorganic capping agents at or near the solvent boundary, where a portion of the organic capping agent may be exchanged/replaced with a portion of the inorganic capping agent. Thus, inorganic capping agents may displace organic capping agents from the surface of semiconductor nanocrystals, and inorganic capping agents may bind to that. This process continues until an equilibrium is established between inorganic capping agents and the free inorganic capping agents. Preferably, the equilibrium favors inorganic capping agents. All the steps described above may be carried out in a nitrogen environment inside a glove box.

Some examples of polar solvents may include 1,3-butanediol, acetonitrile, ammonia, benzonitrile, butanol, dimethylacetamide, dimethylamine, dimethylethylenediamine, dimethylformamide, dimethylsulfoxide (DMSO), dioxane, ethanol, ethanolamine, ethylenediamine, ethyleneglycol, formamide (FA), glycerol, methanol, methoxyethanol, methylamine, methylformamide, methylpyrrolidinone, pyridine, tetramethylethylenediamine, triethylamine, trimethylamine, trimethylethylenediamine, water, and mixtures thereof. Polar solvents such as FA, spectroscopy grade, and DMSO, anhydrous, 99.9% may be supplied by Sigma-Aldrich Co. LLC. Suitable colloidal stability of semiconductor nanocrystals dispersions is mainly determined by a solvent dielectric constant, which may range between about 106 to about 47, with about 106 being preferred.

Examples of non-polar or organic solvents may include tertiary-Butanol, pentane, pentanes, cyclopentane, hexane, hexanes, cyclohexane, heptane, octane, isooctane, nonane, decane, dodecane, hexadecane, benzene, 2,2,4-trimethylpentane, toluene, petroleum ether, ethyl acetate, diisopropyl ether, diethyl ether, carbon tetrachloride, carbon disulfide, and mixtures thereof. Other examples may include alcohol, hexadecylamine (HDA), hydrocarbon solvents at high temperatures.

The purification of inorganic capped semiconductor nanocrystals may require an isolation procedure, such as the precipitation of inorganic product. That precipitation permits one of ordinary skill to wash impurities and/or unreacted materials out of the precipitate. Such isolation may allow for the selective application of photocatalytic capped colloidal nanocrystals.

Preferred inorganic capping agents for photocatalytic capped colloidal nanocrystals may include chalcogenides, and zintl ions, where zintl ions refers to homopolyatomic anions and heteropolyatomic anions that have intermetallic bonds between the same or different metals of the main group, transition metals, lanthanides, and/or actinides.

Additionally, inorganic capping agents may include transition metal chalcogenides such as tetrasulfides and tetraselenides of vanadium, niobium, tantalum, molybdenum, tungsten, and rhenium, and the tetratellurides of niobium, tantalum, and tungsten. These transition metal chalcogenides may further include the monometallic and polymetallic polysulfides, polyselenides, and mixtures thereof, e.g., MoS(Se4)22−, Mo2S62−, and the like.

Suitable compositions of inorganic capping agents for photocatalytic capped colloidal nanocrystals may also include polyoxometalates and oxometalates, such as tungsten oxide, iron oxide, gallium zinc nitride oxide, bismuth vanadium oxide, zinc oxide, titanium dioxide, cadmium sulfide, zinc sulfide, among others.

Method 100 may be adapted to produce a wide variety of photocatalytic capped colloidal nanocrystals. Adaptations of method 100 may include adding two different inorganic capping agents to a single semiconductor nanocrystal, adding two different semiconductor nanocrystals to a single inorganic capping agent, adding two different semiconductor nanocrystals to two different inorganic capping agents, and/or additional multiplicities.

The sequential addition of inorganic capping agents to semiconductor nanocrystals may be possible under the disclosed method 100. Depending, for example, upon concentration, nucleophilicity, bond strength between capping agents and semiconductor nanocrystal, and bond strength between semiconductor nanocrystal face dependent capping agent and semiconductor nanocrystal, inorganic capping of semiconductor nanocrystals may be manipulated to yield other combinations.

Suitable photocatalytic capped colloidal nanocrystals may include ZnS.TiO₂, TiO₂.CuO, ZnS.RuOₓ, ZnS.ReOₓ, among others.

As used here the denotation ZnS.TiO₂ may refer to ZnS semiconductor nanocrystal capped with TiO₂ inorganic capping agent. Charges on inorganic capping agent are omitted for clarity. This nomenclature [semiconductor nanocrystal].[inorganic capping agent] is used throughout this description. The specific percentages of semiconductor nanocrystals and inorganic capping agent may vary between different types of photocatalytic capped colloidal nanocrystal.

### Structure of Photocatalytic Capped Colloidal Nanocrystal

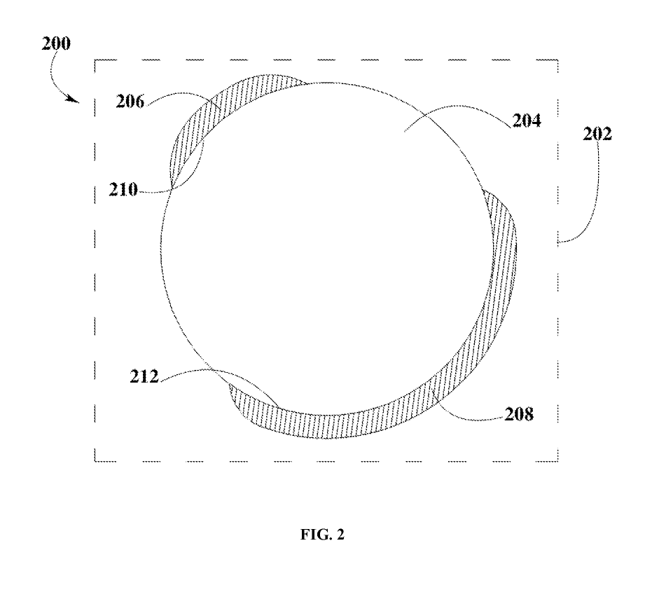

FIG. 2 depicts an illustrative embodiment of spherical configuration 200 of photocatalytic capped colloidal nanocrystal 202 that may include a single semiconductor nanocrystal 204 capped with first inorganic capping agent 206 (reduction photocatalyst) and second inorganic capping agent 208 (oxidative photocatalyst). Semiconductor nanocrystals 204 shown in this embodiment may include face A 210 and face B 212; the bond strength of organic capping agent to face A 210 may be twice that of the bond strength to face B 212. Organic capping agents on face B 212 may be preferably exchanged when employing method 100 for forming photocatalytic capped colloidal nanocrystals 202 described above. Isolation and reaction of this intermediate species, having organic and inorganic capping agents, with second inorganic capping agent 208 may produce photocatalytic capped colloidal nanocrystal 202 with first inorganic capping agent 206 on face B 212 and second inorganic capping agent 208 on face A 210. Alternatively, the preferential binding of inorganic capping agents to specific single semiconductor nanocrystal 204 faces may yield the same result from a single mixture of multiple inorganic capping agents.

As an embodiment, ZnS may be used as semiconductor nanocrystal 204, TiO₂ as first inorganic capping agent 206 and ReO₂ as second inorganic capping agent 208, therefore forming photocatalytic capped colloidal nanocrystal 202 represented as ZnS.(TiO₂;ReO₂).

Another aspect of method 100 is the possibility of a chemical reactivity between first inorganic capping agent 206 and second inorganic capping agent 208. For example, first inorganic capping agent 206 bound to the surface of semiconductor nanocrystal 204 may react with second inorganic capping agent 208. As such, method 100 may also provide for the synthesis of photocatalytic capped colloidal nanocrystal 202 that could not be selectively made from a solution of semiconductor nanocrystal 204 and inorganic capping agents. The interaction of first inorganic capping agent 206 with semiconductor nanocrystal 204 may control both the direction and scope of the reactivity of first inorganic capping agent 206 with second inorganic capping agent 208. Furthermore, method 100 may control the specific areas where first inorganic capping agent 206 may bind to semiconductor nanocrystal 204. The result of the addition of a combined inorganic capping agent capping to semiconductor nanocrystal 204 by other methods may produce a random arrangement of the combined-inorganic capping agent on semiconductor nanocrystal 204.

In addition, the shape of semiconductor nanocrystal 204 may improve photocatalytic activity of semiconductor nanocrystal 204. Changes in shape may expose different facets as reaction sites and may change the number and geometry of step edges where reactions may preferentially take place.

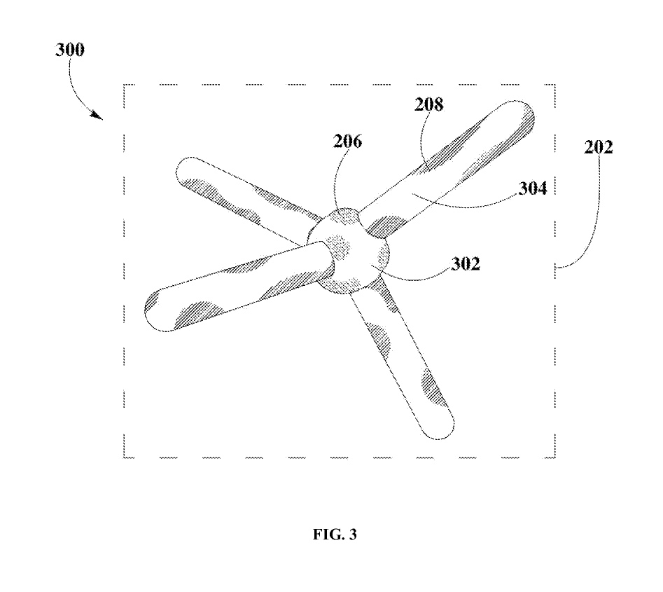

FIG. 3 illustrates an embodiment of tetrapod configuration 300 of photocatalytic capped colloidal nanocrystal 202, that may include first semiconductor nanocrystal 302 capped with first inorganic capping agent 206 and second semiconductor nanocrystal 304 that may be capped second inorganic capping agent 208. As an example, photocatalytic capped colloidal nanocrystal 202 in tetrapod configuration 300 may include (ZnS;TiO₂). (Cu;TiO₂), in which first semiconductor nanocrystal 302 may be ZnS, capped with TiO₂ as first inorganic capping agent 206, while second semiconductor nanocrystal 304 may be Cu, capped with TiO₂ as second inorganic capping agent 208.

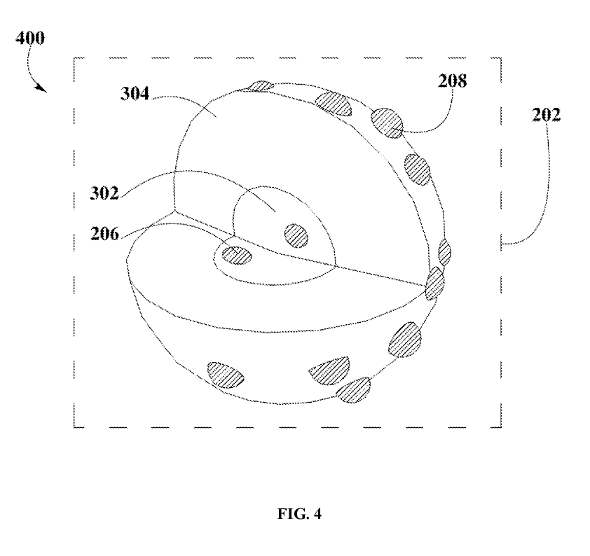

FIG. 4 depicts an illustrative embodiment of core/shell configuration 400 of photocatalytic capped colloidal nanocrystal 202 that may include first semiconductor nanocrystal 302 and second semiconductor nanocrystal 304 that may be capped respectively with first inorganic capping agent 206 and second inorganic capping agent 208. As an example, photocatalytic capped colloidal nanocrystal 202 in core/shell configuration 400 may include (ZnS;Cu).TiO₂, where first semiconductor nanocrystal 302 may be ZnS, while second semiconductor nanocrystal 304 may be Cu, and TiO₂ may be both first inorganic capping agent 206 and second inorganic capping agent 208.

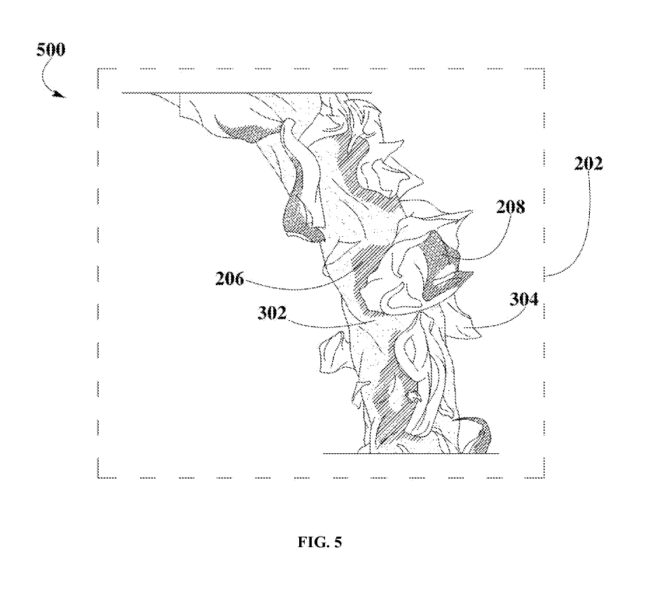

FIG. 5 shows an embodiment of carbon nanotubes configuration 500 of photocatalytic capped colloidal nanocrystal 202, including first semiconductor nanocrystal 302 and second semiconductor nanocrystal 304 capped with first inorganic capping agent 206 and second inorganic capping agent 208, respectively. As an example, photocatalytic capped colloidal nanocrystal 202 in carbon nanotubes configuration 500 may include a carbon nanotube as first semiconductor nanocrystal 302, and graphene foliates as second semiconductor nanocrystal 304; ZnS may be first inorganic capping agent 206 and TiO₂ second inorganic capping agent 208, respectively. Depositing a high density of second semiconductor nanocrystal 304 graphene foliates along the length of aligned first semiconductor nanocrystal 302 carbon nanotube may significantly increase the total charge capacity per unit of nominal area as compared to other carbon nanostructures.

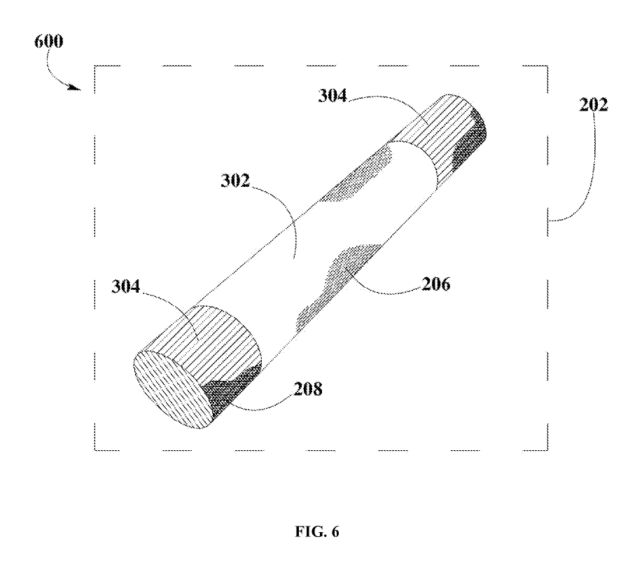

FIG. 6 depicts an embodiment of photocatalytic capped colloidal nanocrystal 202 in nanorod configuration 600. There may be one ZnS region and two Cu regions as first semiconductor nanocrystal 302 and second semiconductor nanocrystal 304, respectively, where first semiconductor nanocrystal 302 may be larger than each of the two second semiconductor nanocrystal 304 of nanorod configuration 600. In other embodiments, the different regions with different materials may have the same lengths, and there can be any suitable number of different regions. The number of regions per nanorod superlattice in nanorod configuration 600 may vary according to the length of the nanorod.

First semiconductor nanocrystal 302 and second semiconductor nanocrystal 304 may be capped with first inorganic capping agent 206 and second inorganic capping agent 208, respectively. First inorganic capping agent 206 may include ReO₂, while W₂O₃ may be employed as second inorganic capping agent 208. Second semiconductor nanocrystal 304 may be placed at the end points of nanorod configuration 600.

The band gap of photocatalytic capped colloidal nanocrystal 202 in nanorod configuration 600 may depend on the size of first semiconductor nanocrystal 302 and second semiconductor nanocrystal 304. Thus, matching the size of the bulk material value for fully converted photocatalytic capped colloidal nanocrystal 202 in nanorod configuration 600 and shifting to higher energy in smaller segments due to quantum confinement.

### Method of Deposition

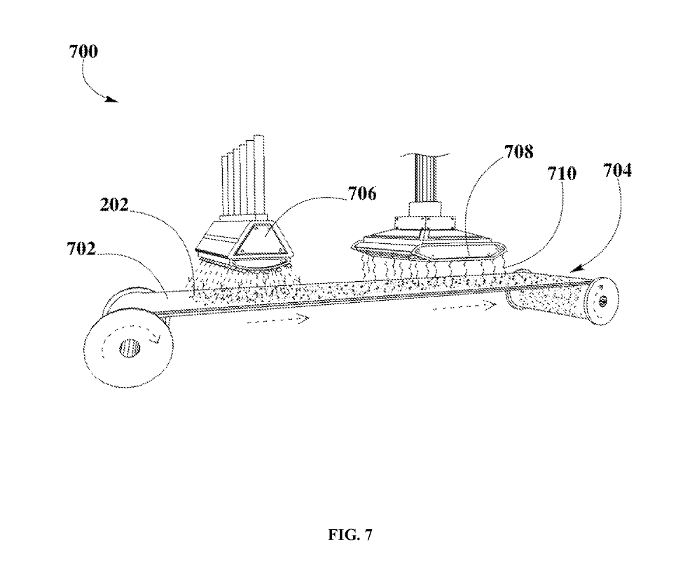

FIG. 7 depicts an embodiment of known in the art spraying deposition and annealing methods 700 used to apply and thermally treat photocatalytic capped colloidal nanocrystal 202 composition on a porous substrate 702. Porous substrate 702 may have a pore size sufficient for a gas (i.e. CO₂, H₂) to pass though at a constant flow rate. In some embodiments, porous substrate 702 may also be optically transparent in order to allow photocatalytic capped colloidal nanocrystal 202 to receive more light. Suitable porous substrate 702 may include glass frits, fiberglass cloth, porous alumina and porous silicon.

Photocatalytic capped colloidal nanocrystal 202 may be applied to suitable porous substrate 702 by different means including plating, chemical synthesis in solution, chemical vapor deposition (CVD), plasma enhanced chemical vapor deposition (PECVD), laser ablation, thermal evaporation, molecular beam epitaxy, electron beam evaporation, pulsed laser deposition (PLD), sputtering, reactive sputtering, atomic layer deposition, spraying deposition and annealing methods and any combinations of those methods. The thickness of photocatalytic capped colloidal nanocrystal 202 can be varied to tune properties of resultant photoactive material 704.

In one embodiment, first inorganic capping agent 206 or second inorganic capping agent 208 may be deposited by spraying device 706 on porous substrate 702. First inorganic capping agent 206 or second inorganic capping agent 208 may be precursors to inorganic materials (matrices) and low-temperature thermal treatment of first inorganic capping agent 206 or second inorganic capping agent 208. Employing convection heater 708 may provide a method to produce crystalline films from photocatalytic capped colloidal nanocrystal 202. The thermal treatment of photocatalytic capped colloidal nanocrystal 202 may yield, for example, ordered arrays of semiconductor nanocrystal 204 within an inorganic matrix, hetero-alloys, or alloys. In at least one embodiment, convection heat 710 applied over photocatalytic capped colloidal nanocrystal 202 may reach temperatures from about 180° C. to about 350° C.

As a result of spraying deposition and annealing methods 700, photoactive material 704 may be formed. Photoactive material 704 may then be cut into films to be used in subsequent carbon dioxide reduction methods.

According to another embodiment, deposition on porous substrate 702 may not be needed. Accordingly, photocatalytic capped colloidal nanocrystal 202 may be deposited into a crucible and then annealed. Solid photocatalytic capped colloidal nanocrystal 202 may then be ground into particles and sintered to form photoactive material 704 that may be deposited on a suitable surface where photoactive material 704 may adhere. In another embodiment, ground particles may be used directly as photoactive material 704.

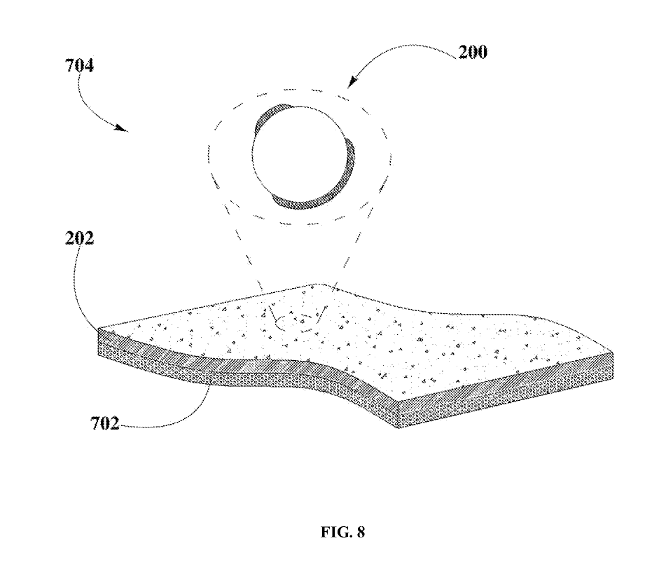

FIG. 8 shows photoactive material 704 including treated photocatalytic capped colloidal nanocrystal 202 in spherical configuration 200 over porous substrate 702. Photocatalytic capped colloidal nanocrystal 202 in photoactive material 704 may also exhibit tetrapod configuration 300, core/shell configuration 400, and carbon nanotubes configuration 500, among others.

Performance of photoactive material 704 may be related to light absorbance, charge carrier mobility and energy conversion efficiency. In order to measure such performance, devices such as transmission electron microscopy (TEM) and energy dispersive X-ray (EDX), among others, may be utilized.

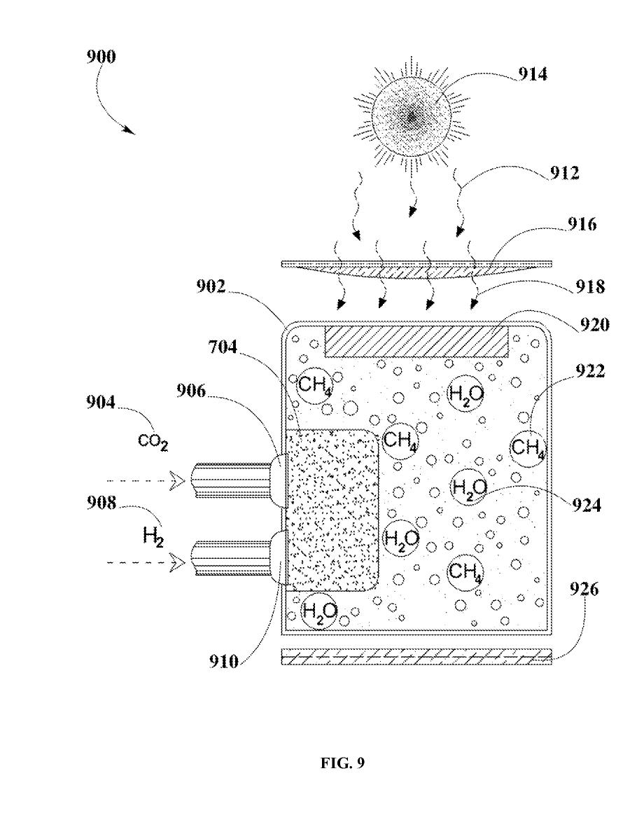

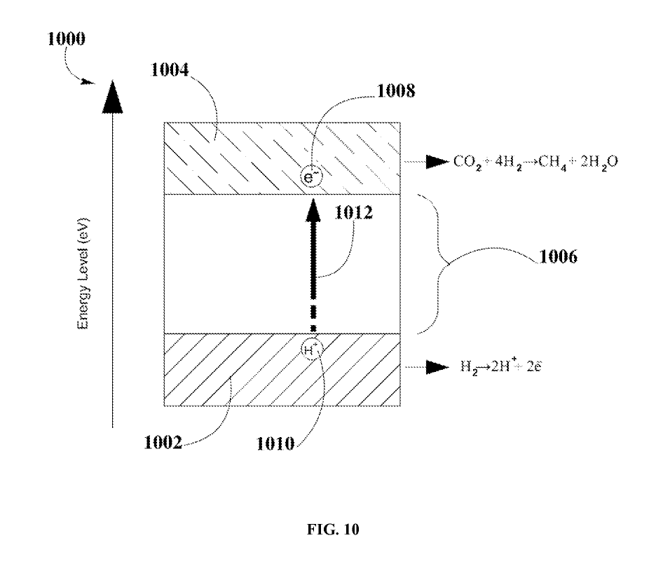

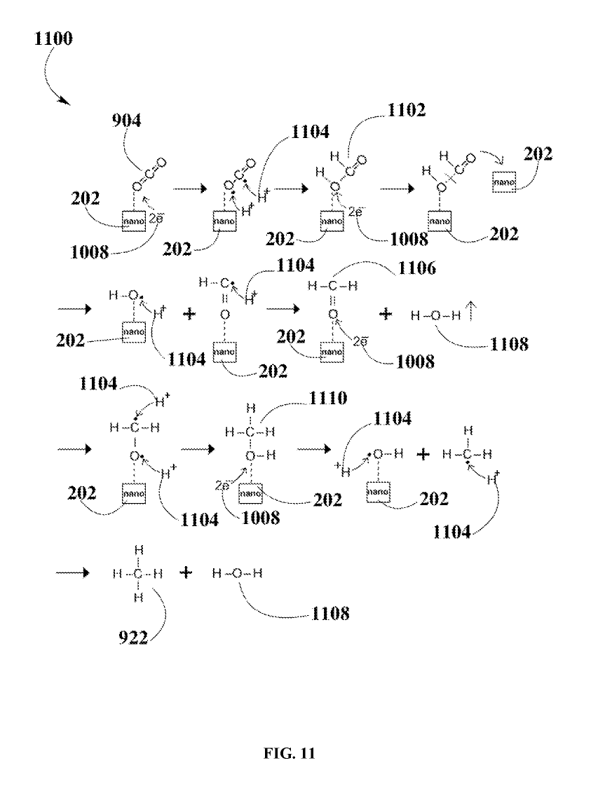

FIG. 9 represents carbon dioxide reduction process 900, carried out within reaction vessel 902 and employing photoactive material 704. Process 900 is driven by light 912 from light source 914, which is intensified by light intensifier 916, and then directed into reaction vessel 902 through window 920. Intensifier 916 is be a solar concentrator, such as a parabolic solar concentrator. Carbon dioxide gas 904 and hydrogen gas 908 are injected into reaction vessel 902 by inlet lines 906 and 910, respectively. The two gases pass through photoactive material 704 prior to entering reaction vessel 902. Intensified light 918 reacts with photoactive material 704 to produce charge separation (explained in FIG. 10) in the boundary of photoactive material 704. Carbon dioxide 904 is reduced and hydrogen gas 908 is oxidized by a series of reactions, ultimately producing methane molecules 922 and water vapor 924 are produced, as discussed in connection with FIG. 11.

A number of variations to the structure shown in FIG.11 can be envisioned by those having ordinary skill in the art. For example, solar reflector 926 may be positioned at the bottom or any side of reaction vessel 902 to reflect intensified light 918 back to reaction vessel 902 to re-utilize it. Also, or more walls of reaction vessel 902 may be formed of glass or other transparent material, so that intensified light 918 may enter reaction vessel 902 to react with photoactive material 704. Alternatively, reaction vessel 902 may have one transparent side to allow intensified light 918 to enter, while the other sides may have a reflective interior surface to reflect the majority of intensified light 918 into photoactive material 704.

A preferable light source 914 to provide light 912 for carbon dioxide reduction process 900 may be sunlight, containing infrared, ultraviolet and visible light. Sunlight may be diffused, direct light, or both, or it may be filtered or unfiltered, modulated or unmodulated, attenuated or unattenuated. Light intensifier 916 may employ any suitable combination of lenses, mirrors, waveguides, or other optical devices, to increase the intensity of light 912. Intensification occurs primarily at wavelengths from about 300 to about 1500 nm, and most particularly from about 300 nm to about 800 nm, intensifying light 912 by a factor greater than about 2, more preferably a factor greater than about 10, and most preferably a factor greater than about 25.

FIG. 10 illustrates charge separation process 1000 that may occur during carbon dioxide reduction process 900. The energy difference between valence band 1002 and conduction band 1004 of semiconductor nanocrystal 204 is known as a band gap 1006. Valence band 1002 refers to outermost electron 1008 shell of atoms in semiconductor nanocrystal 204 and insulators in which electrons 1008 are too tightly bound to the atom to carry electric current, while conduction band 1004 refers to the band of orbitals that are high in energy and are generally empty. Band gap 1006 of semiconductor nanocrystal 204 should be large enough to drive carbon dioxide reduction process 900 reactions but small enough to absorb a large fraction of light 912 wavelengths. Band gap 1006 of photocatalytic capped colloidal nanocrystal 202 employed in the reduction of carbon dioxide 904 should be at least 1.33 eV, which corresponds to absorption of solar photons of wavelengths below 930 nm. Considering the energy loss associated with entropy change (87 J/mol·K) and other losses involved in carbon dioxide 904 reduction (forming methane molecule 922 and water vapor 924), band gap 1006 between about 2 and about 2.4 eV may be preferred. The manifestation of band gap 1006 in optical absorption is that only photons with energy larger than or equal to band gap 1006 are absorbed.

When semiconductor nanocrystal 204 in photoactive material 704 are irradiated with photons having a level of energy greater than band gap 1006 of photoactive material 704, electrons 1008 may be excited from valence band 1002 into conduction band 1004, leaving holes 1010 behind in valence band 1002. Excited electrons 1008 may reduce carbon dioxide 904 molecules into methane molecule 922, while holes 1010 may oxidize hydrogen gas 908 molecules. Oxidized hydrogen molecules may react with carbon dioxide 904 and form water and methane molecule 922 via a series of reactions. Electrons 1008 may acquire energy corresponding to the wavelength of absorbed light 912. Upon being excited, electrons 1008 may relax to the bottom of conduction band 1004, which may lead to recombination with holes 1010 and therefore to an inefficient charge separation process 1000.

Following photo-excitation 1012 to conduction band 1004, electron 1008 can quickly move to the acceptor state of first inorganic capping agent 206 and hole 1010 can move to the donor state of second inorganic capping agent 208, preventing recombination of electrons 1008 and holes 1010. First inorganic capping agent 206 acceptor state and second inorganic capping agent 208 donor state lie energetically between the limits of band gap 1006 and the redox potentials of the hydrogen oxidation and carbon dioxide 904 reduction reactions. By being more stable to recombination in the donor and acceptor states, charge carriers may be stored for use in redox reactions required for a more efficient charge separation process 1000, and hence, a more productive carbon dioxide reduction process 900.

Referring now to FIG. 11, chemical reactions 1100 describe carbon dioxide reduction process 900, in which carbon dioxide 904 is photocatalytically reduced to methane molecule 922 by means of photocatalytic capped colloidal nanocrystal 202 in photoactive material 704. Electrons 1008 may be obtained from the reaction between carbon dioxide 904 and photocatalytic capped colloidal nanocrystal 202 in photoactive material 704, and hydrogen atoms 1104 may be obtained from hydrogen gas 908. Beginning from adsorbed carbon dioxide 904, formic acid 1102 (HCOOH) is formed (1) by accepting two electrons 1008 and adding two hydrogen atoms 1104. Then, formaldehyde 1106 (HCHO) and water molecules 1108 are formed (2) from the reduction of formic acid 1102 by accepting two electrons 1008 and adding two hydrogen atoms 1104. Subsequently, methanol 1110 (CH₃OH) is formed (3) when formaldehyde 1106 accepts two electrons 1008 and two hydrogen atoms 1104 are added to formaldehyde 1106. Finally, methane molecule 922 is formed (4) when methanol 1110 accepts two electrons 1008 and two hydrogen atoms 1104 are added to methanol 1110. In addition, water is formed as a byproduct of the reaction. Chemical reactions 1100 may be summarized by the following equations:

CO₂+2H⁺+2e⁻→HCOOH (1)

HCOOH+2H⁺+2e⁻→HCHO+H₂O (2)

HCHO+2H⁺+2e⁻→CH₃OH (3)

CH₃OH+2H⁺+2e⁻→CH₄+H₂O (4)

The reduction of carbon dioxide 904 to methane molecule 922 requires eight electrons 1008 for the reduction of the chemical state of carbon from C (4+) to C (4−) for the production of each methane molecule 922. Taken as a whole, eight hydrogen atoms 1104 and eight electrons 1008 progressively transfer to one adsorbed carbon dioxide 904 molecule, producing one methane molecule 922. Similarly, oxygen released from carbon dioxide 904 may react with free hydrogen radicals and form water vapor 924 molecules.

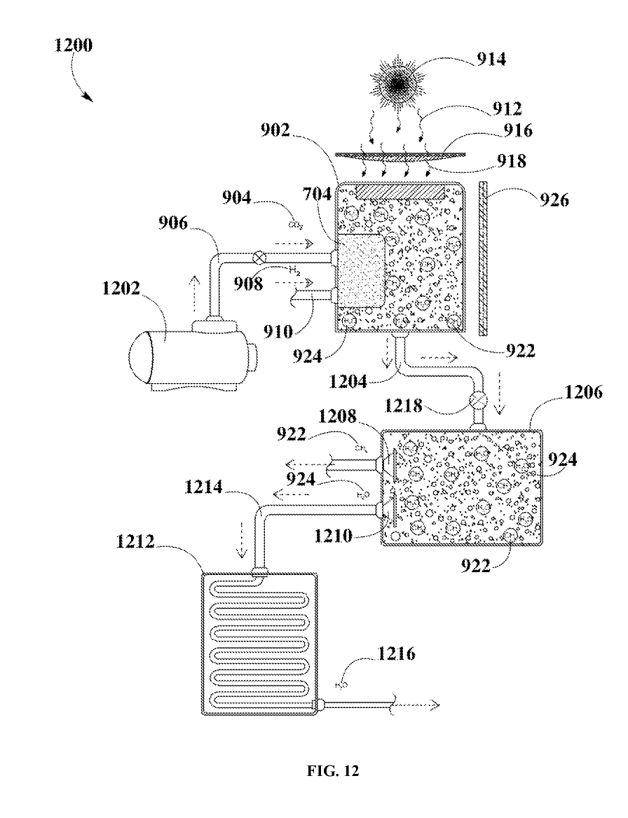

FIG. 12 depicts carbon dioxide reduction system 1200 employing carbon dioxide reduction process 900. Carbon dioxide reduction system 1200 may operate in conjunction with a combustion system that produces carbon dioxide 904 as a byproduct. C This system may be employed to take advantage of carbon dioxide 904 produced by one or more boilers 1202 during a manufacturing process. Boiler 1202 may be connected to reaction vessel 902 by inlet line 906 to allow a continuous flow of carbon dioxide 904 gas. Subsequently, carbon dioxide 904 may pass through photoactive material 704. Similarly, hydrogen gas 908 may also be injected into reaction vessel 902 via inlet line 910. Optionally, a heater (not shown) may be employed to increase the temperature in reaction vessel 902.

Following chemical reactions 1100 described above, the produced methane molecule 922 and water vapor 924 may exit reaction vessel 902 through outlet line 1204 and enter collector 1206, where a methane-permeable membrane 1208 and a water vapor permeable membrane 1210 may collect methane molecules 922 and water vapor 924, respectively. In one embodiment, the membranes may be a polymide resin membrane and a polydimethylsiloxane membrane, respectively. The collected methane molecules 922 may be subsequently stored in any suitable storage medium, or the methane may be directly used as fuel by boiler 1202. The collected water vapor 924 may be transferred to water condenser 1212 through outlet line 1214 to obtain liquid water 1216. Valves 1218, pumps or monitoring devices may be added in order to measure and regulate pressure and/or flow rate. The flow rate of carbon dioxide 904 and hydrogen gas 908 into reaction vessel 902 may be adjusted depending on reaction time between carbon dioxide 904, hydrogen gas 908, and photoactive material 704. Optionally, a gas sensor device (not shown) may be attached to collector 1206 to identify any methane molecule 922 leakage. Liquid water 1216 may be employed for different purposes in the manufacturing process.

While various aspects and embodiments have been disclosed, other aspects and embodiments are contemplated. The various aspects and embodiments disclosed are for purposes of illustration and are not intended to be limiting, with the true scope and spirit being indicated by the following claims.

I claim:

1. A photocatalytic capped colloidal nanocrystal, comprising
first and second semiconductor nanocrystals, formed as a nanorod, the first semiconductor nanocrystal nanorod regions lying at the ends of the nanorod, and the second semiconductor nanocrystal nanorod region disposed between the first semiconductor nanocrystal regions;
a first inorganic capping agent overlying at least a portion of the first semiconductor nanocrystal; and
a second inorganic capping agent overlying at least a portion of the second semiconductor nanocrystal.

2. The photocatalytic capped colloidal nanocrystal of claim 1, wherein the first semiconductor nanocrystal is Cu and the second semiconductor nanocrystal is ZnS.

3. The photocatalytic capped colloidal nanocrystal of claim 1, wherein the second semiconductor nanocrystal region is longer than each first semiconductor nanocrystal region.

2. The photocatalytic capped colloidal nanocrystal of claim 1, wherein the second inorganic capping agent is ReO₂.

3. The photocatalytic capped colloidal nanocrystal of claim 1, wherein the first inorganic capping agent is W₂O₃.

4. A method for reducing carbon dioxide, comprising:
introducing carbon dioxide gas and hydrogen gas into a reaction vessel;
shining light onto a photoactive material within the reaction vessel, the light having sufficient energy to produce charge separation within the photoactive material;
passing the carbon dioxide gas through the photoactive material to react the carbon dioxide with the photoactive material, initiating reduction of the carbon dioxide to methane and oxidation of the hydrogen to water.

5. The method of claim 4, wherein the photoactive material is a photocatalytic capped colloidal nanocrystal.

6. The method of claim 5, wherein the photocatalytic capped colloidal nanocrystal includes at least one semiconductor nanocrystal and at least one inorganic capping agent.

7. The method of claim 5, wherein the photocatalytic capped colloidal nanocrystal includes first and second semiconductor nanocrystals and first and second inorganic capping agents, the first and second capping agents overlying at least portions of the first and second semiconductor nanocrystals, respectively.

8. The method of claim 7, wherein one of the first or second capping agents is a reduction photocatalyst and the other of the first or second capping agents is and oxidation photocatalyst.

9. The method of claim 4, wherein the shining includes intensifying light originating at a light source.

10. The method of claim 4, wherein the light is sunlight.

11. The method of claim 10, wherein the light has a wavelength from about 300 nm to about 1500 nm.

12. The method of claim 10, wherein the light has a wavelength from about 300 nm to about 800 nm.

13. The method of claim 9, wherein the intensifying includes passing the light through one or more of fences, mirrors, waveguides, or optical devices.

14. The method of claim 4, wherein the photoactive material is carried on a porous substrate.

15. The method of claim 14, wherein the porous substrate has a pore size sufficient to admit CO₂ and H₂ gas.

16. The method of claim 4, further comprising reflecting light emerging from the reaction vessel back into the vessel.
